SEQUENCE LISTING (7 sequences)

SEQ ID NO: 1 - protein, 19 aa, Homo sapiens
TFFYGGSRGKRNNFKTEEY

SEQ ID NO: 2 - protein, 252 aa, Homo sapiens
DIQSFSFKNFNSSSFILQGDATVSSSKLRLTKVKGNGLPTLSSLGRAFYSSPIQIYDKSTGAVASWATSFTANIFAPNKSSSADGIAFALVPVGSEPKSNSGFLGVFDSDVYDNSAQTVAVEFDTFSNTDWDPTSRHIGIDVNSIKSIRTASWGLANGQNAEILITYNAATSLLVASLVHPSRRTSYIVSERVDITNELPEYVSIGFSATTGLSEGYTETHDVLSWSFASKLPDDSTTEPLDIASYLVRNVL

SEQ ID NO: 3 - protein, 273 aa, Homo sapiens
MKPFCVFLTFFLLLAASSKKVDSAETVSFNFNSFSEGNPAINFQGDVTVLSNGNIQLTNLNKVNSVGRVLYAMPVRIWSSATGNVASFLTSFSFEMKDIKDYDPADGIIFFIAPEDTQIPAGSIGGGTLGVSDTKGAGHFVGVEFDTYSNSEYNDPPTDHVGIDVNSVDSVKTVPWNSVSGAVVKVTVIYDSSTKTLSVAVTNDNGDITTIAQVVDLKAKLPERVKFGFSASGSLGGRQIHLIRSWSFTSTLITTTRRSIDNNEKKIMNMASA

SEQ ID NO: 4 - protein, 253 aa, Homo sapiens
AETVSFSWNKFVPKQPNMILQGDAIVTSSGKLQLNKVDENGTPKPSSLGRALYSTPIHIWDKETGSVASFAASFNFTFYAPDTKRLADGLAFFLAPIDTKPQTHAGYLGLFNENESGDQVVAVEFDTFRNSWDPPNPHIGINVNSIRSIKTTSWDLANNKVAKVLITYDASTSLLVASLVYPSQRTSNILSDVVDLKTSLPEWVRIGFSAATGLDIPGESHDVLSWSFASNLPHASSNIDPLDLTSFVLHEAI

SEQ ID NO: 5 - protein, 251 aa, Homo sapiens
FNLPNFWSDVKDNIIFQGDANTTAGTLQLCKTNQYGNPLQYRAGRALYSDPVQLWDNKTGSVASFYTEFTFFLKITGDGPADGLAFFLAPPDSDVKDAGAYLGLFNKSTATQPSKNQVVAVEFDTWKNTDFPEPSYRHIGINVNSIVSVATKRWEDSDIFSGKIATARISYDGSAKILTVVLSYPDGADYILSHSVDLSKNLPNPIRVGISASTGANQFLTVYVLSWRFSSALQSTSVNAAMGPEIIRTVV

SEQ ID NO: 6 - protein, 51 aa, Homo sapiens
MAKSTAKPNFSLLLPILISLFLFQLNRVKSADTVCFTSTSFGKDVSDLTLQ

SEQ ID NO: 7 - protein, 242 aa, Homo sapiens
VETISFSFSEFEPGNNDLTLQGAAIITQSGVLQLTKINQNGMPAWDSTGRTLYTKPVHIWDMTTGTVASFETRFSFSIEQPYTRPLPADGLVFFMGPTKSKPAQGYGYLGVFNNSKQDNSYQTLAVEFDTFSNPWDPPQVPHIGIDVNSIRSIKTQPFQLDNGQVANVVIKYDASSKILLAVLVYPSSGAIYTIAEIVDVKQVLPEWVDVGLSGATGAQRDAAETHDVYSWSFHASLPETND

(19) United States
(12) Patent Application Publication
(10) Pub. No.: US 2014/0010886 A1
(43) Pub. Date: Jan. 9, 2014

(54) COMPOSITIONS COMPRISING SACCHARIDE BINDING MOIETIES AND METHODS FOR TARGETED THERAPY

(71) Applicants: David Scott Wilson, Broken Arrow, OK (US); Rodney J. Nash, Atlanta, GA (US); Prasanthi Chappa, Atlanta, GA (US); Constantinos G. Hadjipanayis, Decatur, GA (US)

(72) Inventors: David Scott Wilson, Broken Arrow, OK (US); Rodney J. Nash, Atlanta, GA (US); Prasanthi Chappa, Atlanta, GA (US); Constantinos G. Hadjipanayis, Decatur, GA (US)

(73) Assignees: GEORGIA TECH RESEARCH CORPORATION, Atlanta, GA (US); EMORY UNIVERSITY, Atlanta, GA (US)

(21) Appl. No.: 14/006,271

(22) PCT Filed: Apr. 4, 2012
(86) PCT No.: PCT/US12/32072

§ 371 Date: Sep. 19, 2013

Related U.S. Application Data
(60) Provisional application No. 61/472,797, filed on Apr. 7, 2011.

(51) Int. Cl. A61K 47/48; A61N 5/10

(52) U.S. Cl. CPC A61K 47/48261; A61N 5/10
USPC 424/491; 524/498; 424/78.3; 600/1

(57) ABSTRACT

The disclosure relates to uses of saccharide binding moieties, e.g., lectins for targeting cells, typically cancer stem cells. In certain embodiments, the disclosure relates to conjugates comprising: a) a saccharide binding moiety; b) a polymer; and c) a therapeutic agent; wherein the saccharide binding protein is covalently attached to the polymer.

### CROSS REFERENCE TO RELATED APPLICATIONS

This application claims priority to U.S. Provisional Application No. 61/472,797 filed Apr. 7, 2011, hereby incorporated by reference in its entirety.

### BACKGROUND

Current treatment for glioblastoma multiforme (GBM), the most common type of primary brain tumors, remains suboptimal. The treatment can involve chemotherapy, radiotherapy, and surgery, all of which typically do not provide a cure. Surgical removal of tumors often results in re-growth, and using traditionally chemotherapy regiments for treating GBM is believed to be thwarted by the blood-brain barrier. Thus, there is a need for identifying methods preventing tumor growth and improving circulation of chemotherapeutic agents in the brain.

Cancer stem cells (CSCs) are cancer cells that possess some characteristics associated with normal stem cells such as the ability to differentiate; however, CSCs are tumorigenic. If cancer treatments of do not properly destroy enough CSCs after surgery, the tumor will reappear. There is a risk that post-operative chemotherapy will leave only chemotherapy-resistant CSCs and the ensuing tumor will be resistant to future chemotherapy. It becomes more and more difficult to remove the tumor without conferring resistance and leaving CSCs behind for the tumor to reappear. Thus, it is one object of the disclosure to identify compositions and methods for improving the identification and removal of GBM CSCs after surgical intervention.

Since the expression of CD133 is a marker of tumor cells with stem-cell ability, agents aimed at targeting CD133⁺ tumor cells have been disclosed. For example, Smith et al., Br J Cancer, 2008, 99:100-9, disclose CD133 antibody-drug conjugates for use in hepatocellular and gastric cancers. Chearwae & Bright, Br J Cancer, 2008, 99, 2044-2053, disclose that PPARγ agonists inhibit growth and expansion of CD133⁺ brain tumor stem cells.

GBM brain tumor stem cells have been identified through their expression of CD133; however CD133 is also a marker expressed in non-malignant neural progenitor cells. Using CD133 exclusively as a marker for GBM tumor derived CSCs (GBM-CSCs) is problematic because it is not consistently expressed in all GBMs and CD133-negative cells have been shown to give rise to tumors in transplant assays. See Lathia et al., Stem Cell Rev., 2010, 7(2) 227-237.

The lectin dolichos biflorus agglutinin (DBA), which recognizes α-N-acetylgalactosamine (GalNAc), was used to characterize mouse embryonic stem cells (mESC) cultures by nondestructive measures. Nash et al., Stem Cells, 2007, 25, 974-982. Presentation of GalNAc on the mESC surface is rapidly down-regulated during differentiation preceding that of know protein markers of pluripotency. Lectins have also been used to investigate metastatic processes in cancer as well as to document the repertoire of glycoepitopes on the surface of embryonal carcinoma cells. See, e.g., Venable et al., BMC Dev Biol., 2005, 5, 15 and Plattner et al., Eur J Pharm Biopharm., 2008, 70, 572-576.

Xin et al., International Journal of Pharmaceutics, 2010, 402, 238-247 disclose an anti-tumor effect of paclitaxel (PTX)-loaded methoxy poly(ethylene glycol)-poly(ε-caprolactone) nanoparticles (MPEG-NP/PTX) against glioblastoma multiforme (GBM). Xin et al., Biomaterials, 2011, 1-13, have also disclosed peptide-conjugated poly(ethylene glycol)-co-poly(e-caprolactone) nanoparticles act as a targeting drug delivery system for brain glioma.

References cited herein are not an admission of prior art.

### SUMMARY

The disclosure relates to uses of saccharide binding moieties and proteins, e.g., lectins for targeting cells, typically cancer stem cells. In certain embodiments, the disclosure contemplates conjugates comprising a saccharide binding moiety and a therapeutic agent optionally containing a biodegradable linker. In certain embodiments, the disclosure relates to conjugates comprising: a) a saccharide binding moiety such as a protein; b) a polymer; and c) a therapeutic agent; wherein the saccharide binding moiety is covalently attached to the polymer. The saccharide binding moiety may be a lectin selected from the group consisting of concanavalin A (CON A), dolichos biflorus agglutinin (DBA), peanut agglutinin (PNA), ricinus communis agglutinin I (RCA 120), soybean agglutinin (SBA), ulex europaeus agglutinin I (UEA I), wheat germ agglutinin (WGA), griffonia simplicifolia lectin I (GSL I), lens culinaris agglutinin (LCA), phaseolus vulgaris erythroagglutinin (PHA-E), phaseolus vulgaris leucoagglutinin (PHA-L), pisum sativum agglutinin (PSA), griffonia simplicifolia lectin II (GSL II), datura stramonium lectin (DSL), erythrina cristagalli lectin (ECL), Jacalin, lycopersicon esculentum lectin (LEL), solanum tuberosum lectin (STL), and vicia villosa lectin (VVA) or variant, portions, or derivatives thereof provided the saccharide binding protein binds a saccharide or polysaccharide selected from the group consisting of α- or β-linked N-acetylgalactosamine, branched and terminal a-linked mannose, a-linked N-acetylgalactosamine, galactosyl (β-1,3)N-acetylgalactosamine, oligosaccharides ending in galactose, terminal α- or β-linked N-acetylgalactosamine, α-linked fucose residues, terminal N-acetylglucosamine or chitobiose, α-N-acetylgalactosamine and α-galactose residues, α-linked mannose residues, galactose, α-linked mannose residues, α- or β-linked N-acetylglucosamine, N-acetyllactosamine, galactosyl (β-1,4)N-acetylglucosamine, galactosyl(β-1,3)N-acetylgalactosamine, N-acetylglucosamine, N-acetylglucosamine and N-acetylmuramic acid. Other saccharide binding moieties to these specific saccharides are also contemplated.

In certain embodiments, the polymer forms a particle comprising a biodegradable barrier. In certain embodiments, the polymer comprises lactone containing monomers and ethylene glycol containing monomers. In certain embodiments, the therapeutic agent is an anticancer agent.

In certain embodiments, polymer surrounds the therapeutic agent, or the therapeutic agent is conjugated to the polymer through a biodegradable bond.

In certain embodiments, the therapeutic agent may be cetuximab, temozolomide, bevacizumab, doxorubicin, hydroxydaunorubicin, bleomycin, dactinomycin, vinblastine, dacarbazine, mechlorethamine, cyclophosphamide, etoposide, teniposide, vincristine, prednisone, a platinum agent (e.g., cisplatin, carboplatin, oxaliplatin), fluorouracil, folinic acid, carmustine, rituximab, methotrexate, procarbazine, epirubicin, irinotecan, ifosfamide, chlorambucil, lomustine, leucovorin, fludarabine, thalidomide, dexamethasone, docetaxel, anastrozole, topotecan, combretastatin, or combretastatin A-4 phosphate. In certain embodiments, the therapeutic agent stimulates the nuclear receptor peroxisome proliferator-activated receptor gamma (PPAR-γ) such as pioglitazone and rosiglitazone.

In certain embodiments, the conjugate further comprises a molecule that facilitated transfer across the blood brain barrier, e.g., a ligand of low-density lipoprotein receptor-related protein wherein the ligand is attached to the polymer. In certain embodiments, the ligand of low-density lipoprotein receptor-related protein is a polypeptide of TFFYGGSRGKRNNFKTEEY (SEQ ID NO:1).

In certain embodiments, the disclosure relates to compositions comprising conjugates comprising a saccharide binding moiety or protein and a polymer wherein the saccharide binding moiety or protein is covalently attached to the polymer wherein the polymer surrounds a metal particle.

In certain embodiments, the disclosure relates to compositions comprising conjugates comprising: a) a saccharide binding moiety or protein; b) a polymer; and c) a therapeutic agent; wherein the saccharide binding moiety or protein is covalently attached to the polymer wherein the polymer surrounds a particle. In certain embodiments, the particle has a diameter of between about 200 and 5 nm. In certain embodiments, the particle is metal particle, e.g., an iron oxide particle, elemental iron coated with iron oxide, gold particle, silver particle, a quantum dot, or bismuth encapsulated in a phospholipid core. In certain embodiments, the therapeutic agent is an EGFR antibody such as cetuximab or a HER-2 antibody such as trastuzumab and the saccharide binding protein is DBA.

In certain embodiments, the disclosure relates to methods of treating cancer comprising administering a composition comprising a conjugate disclosed herein to a subject diagnosed with cancer. Typically, the subject is diagnosed with a brain tumor and has previously undergone surgical removal of a brain tumor or radiation therapy. It is contemplated that administered of the composition may be in combination with radiation therapy or the composition is administered orally, by injection, convection enhance delivery, or intracerebrally.

In certain embodiments, the disclosure relates to methods of purifying cells comprising mixing lectins disclosed herein with cells under conditions that an isolated composition of cells is formed.

In certain embodiments, the disclosure relates to methods of detecting cancer stem cells by assaying a sample of cells using lectins disclosed herein. It is contemplated that lectins may be mixed with a sample comprising cells and the binding of certain lectins are measured to indicate the presence or absence of caner stem cells. This information is then reported to the subject and correlated to an appropriate therapeutic strategy, e.g., drug regiment, surgery, or radiotherapy.

### BRIEF DESCRIPTION OF THE FIGURES

FIG. 1A shows a schematic representation of CD133 positive cell sorting.

FIG. 1B shows dispersal and culturing of CD133 positive sorted cells.

FIG. 1C shows differentiation of CD133 positive cells.

FIG. 1D schematically illustrates CD133.

FIG. 2A shows data on the differentiation of CD133 positive cells.

FIG. 2B shows data on a dated population of lectin labeled CD133 positive cells at day 1 and day 12.

FIG. 2C shows data on the topographical display of lectin labeled CD133 positive cells at day 1 and day 12.

FIG. 3A shows a population of lectin labeled CD133 positive cells at day 1 and day 12.

FIG. 3B shows data on the topographical display of lectin labeled CD133 positive cells at day 1 and day 12.

FIG. 3C shows data on the CD133 glycosylation, OCT 4, and Sox 2 expression at day 1 and day 12 in differentiating CD133 cells.

FIG. 4A shows data on cell sorting of the parent cell population (PC), DBA positive population (SC-DBA), DBA negative population (SC-Neg), and Dual labeled (DBA and Actin positive) population.

FIG. 4B shows a bar graph representation of FIG. 4A.

FIG. 4C shows data on competing sugars (200 mM GalNAc) showing specificity of DBA for GalNAc.

FIG. 5A is a schematic illustration of DBA positive cell sorting.

FIG. 5B shows data on DBA positive and DBA negative cell grown in culture.

FIG. 6 shows data on DBA positive and negative neurosphere differentiation and GFAP labeling. Neurospheres derived from DBA positive and negative sorted cells. After day 12 of differentiation cells were fluorescently labeled for GFAP.

FIG. 7 shows data on the quantitation of lectin binding on DBA positive and DBA negative GBM-CSCs (day 1 and day 12) surfaces with 8 different lectins and 3 stem cells markers.

The percent of cells with specific carbohydrate expression as determined by flow cytometry using 8 different lectins. The data are means+/−SD of 3 independent assays of CTB-1 GBM-CSCs. CTB-1 cell line was stained with CD133, OCT4, and SOX2 antibody in addition to lectin staining Statistical significance (p≦0.05) is indicated by asterisk.

FIG. 8A shows data on cytometric representation of individual populations of DBA, CD133 and CD133-G labeled cells analyzed through flow cytometry.

FIG. 8B shows data on DBA positive sorted cells (day 1 and day 12 of differentiation) labeled for DBA, CD133, and CD133G, analyzed through flow cytometry.

FIGS. 9A and 9B show data on GBM-CSCs at day 1 and day 12 of differentiation labeled for Ki67 and cleaved caspase-3. Bar graph representation of DBA positive and DBA negative sorted cells were grown as neurospheres and differentiated on laminin coated plates for 12 days. Cells were labeled at day 1 and day 12 of differentiation for Ki67 (proliferation) and cleaved caspase-3 (apoptosis) and analyzed using flow cytometry.

FIG. 10 shows a table summarizing data obtained using a panel of 20 fluorescein-isothiocyanate labeled lectins on unfixed (repeated on fixed with the same result) hGBM-CSCs (CTB-1), grown on laminin in CTB-1 neuronal media at day 1 and day 12 in the absence of growth factors.

FIG. 11 illustrates a lectin conjugated to a particle contain a therapeutic agent.

FIG. 12 shows a picture of undifferentiated glioblastoma multiforme derived cancer stem cells that were treated with a nanoparticle coupled to DBA lectin, which recognizes GalNAc. The nanoparticle was incubated with the cells for 24 hours and was internalized and dissolved and diffused within the cell and appear as a reddish color (Rhodamine). The nuclei are stained in blue (DAPI) and the final image is a merger of the two.

FIG. 13 shows a picture of undifferentiated glioblastoma multiforme derived cancer stem cells treated with a nanoparticle coupled to GSL II lectin, which recognizes GlcNAc. After 12 hour of exposure the nanoparticle can be seen in a slightly dissolved state appearing to be reddish. The cell nucleus appears blue (DAPI).

FIG. 14 illustrates an embodiment of the disclosure. Lectins in combination with an anticancer agent such as an EGFR antibody are conjugated to polymer that covers an iron oxide nanoparticle (IONP). In some embodiments, the anticancer agent is conjugated to the particle through a biodegradable linker.

FIG. 15 shows absorbance data in a MTT assay with IONPs-Cetuximab-lectin conjugates on U87wtEGFR glioblastoma cells. The lectins DOLICHOS BIFLORUS AGGLUTININ (DBA) and GRIFFONIA SIMLICIFOLIA II (GSLII) in combination with Cetuximab are conjugated to iron oxide nanoparticles. Row 1, all the way left is a control, in Row 2 IONPs-Cetuximab (0.2 mg/ml) was added, in Row 3 is IONPs-Cetuximab-DBA (0.2 mg/ml), in Row 4 IONPs-Cetuximab-GSLII (0.2 mg/ml), in Row 5 IONPs-DBA (0.2 mg/ml), in Row 6 IONPs-GSLII (0.2 mg/ml), in Row 7 IONPs-IgG-DBA (0.2 mg/ml), in Row 8 IONPs-IgG-GSLII (0.2 mg/ml), in Row 9 IONPs-IgG (0.2 mg/ml), in Row 10 Cetuximab, in Row 11 DBA, and in Row 12 GSLII.

FIG. 16 shows photomicrograph of an H&E and Ki67 stained high grade glioma derived from CTB-1/DBA+GBM Neurospheres implanted in flank of NODSCID mice. (A) solid growth pattern of high-grade glioma with high cell density and perivascular growth pattern. Ki67 labeling index was very high (greater than 40%). (B) High grade malignancy showed features of a glioblastoma including nuclear anaplasia, invasiveness and proliferation of blood vessels.

### DETAILED DISCUSSION

### Terms

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing a amino group with a hydroxyl group. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, “subject” refers to any animal, preferably a human patient, livestock, or domestic pet.

The terms “protein” and “polypeptide” refer to compounds comprising amino acids joined via peptide bonds and are used interchangeably.

As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties.

The term “portion” when used in reference to a protein (as in “a portion of a given protein”) refers to fragments of that protein. The fragments may range in size from four amino acid residues to the entire amino sequence minus one amino acid.

The term “chimera” when used in reference to a polypeptide refers to the expression product of two or more coding sequences obtained from different genes, that have been cloned together and that, after translation, act as a single polypeptide sequence Chimeric polypeptides are also referred to as “hybrid” polypeptides. The coding sequences includes those obtained from the same or from different species of organisms.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, as well as providing an “affinity tag” to allow purification of the recombinant fusion polypeptide from a host cell or from a supernatant or from both. If desired, the fusion partner may be removed from the protein of interest after or during purification.

The terms “variant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. A variant may have “non-conservative” changes (e.g., replacement of a glycine with a tryptophan). Similar minor variations may also include amino acid deletions or insertions (in other words, additions), or both, e.g., chimera or fusion. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “purified” refers to molecules e.g., amino acid sequences, that are removed from their natural environment, isolated or separated. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. The term “sample” is used in its broadest sense. In one sense it can refer to a biopolymeric material. In another sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like.

### Saccharide Binding Moieties

Naturally occurring lectins are sugar binding proteins that play a role in biological recognition. However, a variety of proteins, molecules, and conjugates are capable of preferentially binding a sugar or sugars are contemplated saccharide binding moieties. Saccharide binding proteins include lectins, antibodies, antibody fragments, antibody chimera, antibody mimetics, and aptamers. Non-protein based saccharide binding moieties are also contemplated such as boronic acid containing moieties, such as boronic acid containing bicyclic carbocycles, aromatics, and heterocycles. Certain crown ethers, polyazamacrocycles, cyclodextrins, tripodal polypyridines, and polyhydroxylated steroids are known saccharide binders. See, e.g., Meng et al., Chem Biol Drug Des, 2011, 78:816-825, James et al., Boronic Acids in Saccharide Recognition, 2006, Royal Society of Chemistry, and Schrader & Hamilton, Functional Synthetic Receptors, 2006, John Wiley & Sons, Chapter 2 entitled Carbohydrate Receptors, all hereby incorporated by reference in their entirety.

In certain embodiments, the disclosure contemplates saccharide binding moieties or proteins in any of the disclosed embodiments that are antibodies or fragments or chimera, antibody mimetics, or aptamers or any molecular entity that selectively binds saccharides that are more prevalent on cancer cells.

Numerous methods known to those skilled in the art are available for obtaining antibodies or antigen-binding fragments thereof For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (V_{H}) and light chains (V_{L}) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

Antibody mimetics or engineered affinity proteins are polypeptide based saccharide binding proteins that can specifically bind to saccharides but are not specifically derived from antibody V_{H} and V_{L} sequences. Typically, a protein motif is recognized to be conserved among a number of proteins. One can artificially create libraries of these polypeptides with amino acid diversity and screen them for binding to saccharides through phage, yeast, bacterial display systems, cell-free selections, and non-display systems. See Gronwall & Stahl, J Biotechnology, 2009, 140(3-4), 254-269, hereby incorporated by reference in its entirety. Antibody mimetics include affibody molecules, affilins, affitins, anticalins, avimers, darpins, fynomers, kunitz domain peptides, and monobodies.

Affibody molecules are based on a protein domain derived from staphylococcal protein A (SPA). SPA protein domain denoted Z consists of three α-helices forming a bundle structure and binds the Fc protion of human IgGl. A combinatorial library may be created by varying surface exposed residues involved in the native interaction with Fc. Affinity proteins can be isolated from the library by phage display selection technology. See Orlova et al., Cancer Res., 2007, 67:2178-2186, hereby incorporated by reference in its entirety.

Monobodies, sometimes referred to as adnectins, are antibody mimics based on the scaffold of the fibronectin type III domain (FN3). See Koide et al., Methods Mol. Biol. 2007, 352: 95-109, hereby incorporated by reference in its entirety. FN3 is a 10 kDa, β-sheet domain, that resembles the V_{H} domain of an antibody with three distinct CDR-like loops, but lack disulfide bonds. FN3 libraries with randomized loops have successfully generated binders via phage display (M13 gene 3, gene 8; T7), mRNA display, yeast display and yeast two-hybrid systems. See Bloom & Calabro, Drug Discovery Today, 2009, 14(19-20):949-955, hereby incorporated by reference in its entirety.

Anticalins, sometimes referred to as lipocalins, are a group of proteins characterized by a structurally conserved rigid β-barrel structure and four flexible loops. The variable loop structures form an entry to a ligand-binding cavity. Several libraries have been constructed based on natural human lipocalins, i.e., ApoD, NGAL, and Tlc. See Skerra, FEBS J., 275 (2008), pp. 2677-2683, hereby incorporated by reference in its entirety.

The ankyrin repeat (AR) protein is composed repeat domains consisting of a β-turn followed by two α-helices. Natural ankyrin repeat proteins normally consist of four to six repeats. The ankyrin repeats form a basis for darpins (designed ankyrin repeat protein) which is a scaffold comprised of repeats of an artificial consensus ankyrin repeat domain. Combinatorial libraries have been created by randomizing residues in one repeat domain. Different numbers of the generated repeat modules can be connected together and flanked on each side by a capping repeat. The darpin libraries are typically denoted N×C, where N stands for the N-terminal capping unit, C stands for the C-terminal capping domain and x for the number of library repeat domains, typically between two to four. See Zahnd et al., J. Mol. Biol., 2007, 369:1015-1028, hereby incorporated by reference in its entirety.

Aptamers refer to affinity binding molecules identified from random proteins or nucleic acid libraries. Peptide aptamers have been selected from random loop libraries displayed on TrxA. See Borghouts et al., Expert Opin. Biol. Ther., 2005, 5:783-797, hereby incorporated by reference in its entirety. SELEX (“Systematic Evolution of Ligands by Exponential Enrichment”) is a combinatorial chemistry technique for producing oligonucleotides of either single-stranded DNA or RNA that specifically bind to a target. Standard details on generating nucleic acid aptamers can be found in U.S. Pat. No. 5,475,096, and U.S. Pat. No. 5,270,163. The SELEX process provides a class of products which are referred to as nucleic acid ligands or aptamers, which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Saccharides can serve as targets.

### Lectins Identify Glycan Biomarkers on Glioblastoma Multiforme Derived Cancer Stem Cells

A panel of lectins was used to identify differences in glycan expression found on undifferentiated and differentiated glioma derived cancer stem cells. Fluorescently labeled lectins which recognized N-acetyl galactosamine (GalNAc) and N-acetyl glucosamine (G1cNAc) were shown differentially recognize the surface of the cells, based on their state of differentiation. GalNAc and GlcNAc were shown to be highly expressed on the surface of undifferentiated cells and markedly decreased expression over a 12 day duration of differentiation. Additionally, these glycans were used as tools for sorting cancer stem cell populations from populations positive for differentiation. These results suggest that GalNAc and GlcNAc are novel biomarkers for identifying glioma derived cancer stem cells and can be used to isolate cancer stem cells from unsorted cell population.

Previous studies have shown a higher re-occurrence rate of the same tumor after treatment. Several studies have acknowledged the presence of stem-like cells in brain tumor tissue cultures, which are CD133 positive and have the ability to give rise to all cell types found within the tumor, potentially perpetuating its growth. The existence of a population of precursor cells or cancer stem cells (CSCs), which are resistant to treatment, may promote the re-occurrence of the cancerous legion. It has been discovered that a number of lectins bind undifferentiated GBM tumor derived stem cells. Lectins were used to determine the cell surface glycan expression patterns of CD133⁺ CSCs derived from a human GBM brain tumor via FACS analysis and the CD133− sorted population was used as a control. Five lectins, which recognize GalNAc, and two lectins, which recognize α-N-acetylglucosamine (GlcNAc) were highly reactive towards undifferentiated CD133⁺ GBM tumor derived cells. Presentation of GalNAc and GlcNAc on the cell surface is rapidly diminished during differentiation, in conjunction with GBM-CSC marker CD133, and general stem cell markers (OCT-4 and SOX 2). These findings establish the use of lectins and cell surface glycans (GalNAc and GlcNAc) as nondestructive markers to characterize CSCs derived from GBM brain tumors independently of CD133.

The cell surface glycan expression patterns of the human GBM brain tumor derived CD133⁺ CSC line and negative control cell line (CD133⁻) were first explored by flow cytometry. Afterwards, the lectin DBA was used to separate the two cell lines, based on the recognition of specific cell surface glycosylation, through FACS and magnetic bead separation. This study demonstrates that glycans can be used to separate GBM-derived CSCs from differentiated tumor cells in the presence or absence of the previously defined biomarker CD133⁺.

The resolution of lectins as markers is emphasized by their ability to discriminate between GBM-derived CSCs and differentiated tumor cells. Because differentiated tumor cells have lost their ability to self-renew, lectins are likely to have utility as an indicator of differentiation commitment. The current GBM stem cell marker CD133 cannot be used for this. Interestingly, the only glycans to change substantially on the cell surface during differentiation were GalNAc and GlcNAc. It will be interesting to determine how GalNAc and GlcNAc epitopes are regulated on the surface of GBM-derived CSCs as they differentiate and to determine the identity of proteins and lipids marked with the glycans. The current marker for GBM-derived CSCs, CD133, is highly expressed and upon differentiation it rapidly decreases expression. However, CD133 expression is greatly influenced by various environmental and genetic factors. GBM-derived cells which were negative for CD133 expression have been shown to promote tumor formation. Because GalNAC and GlcNAc epitopes are downregulated concertedly or slightly before CD133, one can define cell populations with greater temporal resolution. In combination with CD133, lectins can be used to define in detail the early stages of differentiation in GBM-derived CSC populations.

GalNAc and GlcNAc are nondectructive markers for GBM derived CSCs cells and are a reliable readout for initial differentiation events, at a level of temporal resolution that was not previously possible. Lectins can be used as recognition tools for these glycan markers which are located on the surface of cells and therefore allowing for cell sorting, culturing and harvesting a specific cell population. This offers an alternative to previous antibody methodologies which are limiting because they require destructive techniques. These markers can also be used for sorting cancer stem cells as well.

### Drug Targeting

Targeting drugs to a specific site provides several advantages over non-specific sites such as preventing side effects of drugs while enhancing uptake by targeted cells. As a result of their specificity, there is a natural basis for the use of lectin-glycan pairs in drug targeting. As mentioned above, GalNAc and GlcNAc are expressed on GBM-derived CSCs. A reverse lectin targeting approach can utilize exogenous lectins as targeting moieties that steer drugs and their delivery system to specific glycans that are expressed on GBM-derived CSC. Lectin targeting was carbohydrate specific with high binding affinity and internalization of cell surface-bound lectin was confirmed. This may show equal promise for the development of functionalized drug delivery systems for site specific therapy targeting GBM-derived CSCs. This approach may prevent tumor reoccurrences which are believe to be associated with the presence of CSCs.

Within certain embodiments it is contemplated that saccharide binding proteins disclosed herein may be conjugated to polymers comprising ethylene glycol monomer and lactone monomers. Xin et al., International Journal of Pharmaceutics, 2010, 402, 238-247, hereby incorporated by reference, disclose an anti-tumor effect of paclitaxel (PTX)-loaded methoxy poly(ethylene glycol)-poly(c-caprolactone) nanoparticles (MPEG-NP/PTX) against glioblastoma multiforme (GBM).

Within certain embodiments it is contemplated that saccharide binding proteins disclosed herein may be conjugated to polymers coating a metal nanoparticle. In one example, quantum dots may be prepared with amphiphilic multidentate ligands and modified with saccharide binding proteins. See Kairdolf et al., J Am Chem Soc, 2008, 130, 12866-12867 and US Published Patent Application Number 2011/0260111. Pegylated colloidal gold and iron oxide nanoparticles are also contemplated. See Kairdolf & Nie, J Am Chem Soc, 2011, 133(19):7268-7271, Qian et al., Nature Biotechnology, 2008, 26, 83-90, Hadjipanayis et al., Cancer Research, 2010, 70(15):6303-6312, and Peng et al., Int J Nanomedicine, 2008, 3(3): 311-321, all hereby incorporated by reference in their entirety.

Contemplated saccharide binding proteins include Dolichos biflorus lectin chain A (SEQ ID NO:2) ADIQSFSFKN FNSSSFILQG DATVSSSKLR LTKVKGNGLP TLSSLGRAFY SSPIQIYDKS 61 TGAVASWATS FTANIFAPNK SSSADGIAFA LVPVGSEPKS NSGFLGVFDS DVYDNSAQTV 121 AVEFDTFSNT DWDPTSRHIG IDVNSIKSIR TASWGLANGQ NAEILITYNA ATSLLVASLV 181 HPSRRTSYIV SERVDITNEL PEYVSIGFSA TTGLSEGYTE THDVLSWSFA SKLPDDSTTE 241 PLDIASYLVR NVL or variants, e.g., conserved variants, or portions thereof that bind α-linked N-acetylgalactosamine

Contemplated saccharide binding proteins include peanut lectin (SEQ ID NO:3) MKPFCVFLTF FLLLAASSKK VDSAETVSFN FNSFSEGNPA 1NFQGDVTVL SNGNIQLTNL 61 NKVNSVGRVL YAMPVRIWSS ATGNVASFLT SFSFEMKDIK DYDPADGIIF FIAPEDTQIP 121 AGSIGGGTLG VSDTKGAGHF VGVEFDTYSN SEYNDPPTDH VGIDVNSVDS VKTVPWNSVS 181 GAVVKVTVIY DSSTKTLSVA VTNDNGDITT IAQVVDLKAK LPERVKFGFS ASGSLGGRQI 241 HLIRSWSFTS TLITTTRRSI DNNEKKIMNM ASA, or variants, e.g., conserved variants, or portions thereof that bind galactosyl (β-1,3)N-acetylgalactosamine

Contemplated saccharide binding proteins include soybean lectin (SEQ ID NO:4) AETVSFSWNK FVPKQPNMIL QGDAIVTSSG KLQLNKVDEN GTPKPSSLGR ALYSTPIHIW 61 DKETGSVASF AASFNFTFYA PDTKRLADGL AFFLAPIDTK PQTHAGYLGL FNENESGDQV 121 VAVEFDTFRN SWDPPNPHIG INVNSIRSIK TTSWDLANNK VAKVLITYDA STSLLVASLV 181 YPSQRTSNIL SDVVDLKTSL PEWVRIGFSA ATGLDIPGES HDVLSWSFAS NLPHASSNID 241 PLDLTSFVLH EAI or variants, e.g., conserved variants, or portions thereof that bind terminal α- or β-linked N-acetylgalactosamine.

Contemplated saccharide binding proteins include isolectin I-A of Griffonia (Bandeiraea) simplicifolia (SEQ ID NO:5) FNLPNFWSDV KDNIIFQGDA NTTAGTLQLC KTNQYGNPLQ YRAGRALYSD PVQLWDNKTG 61 SVASFYTEFT FFLKITGDGP ADGLAFFLAP PDSDVKDAGA YLGLFNKSTA TQPSKNQVVA 121 VEFDTWKNTD FPEPSYRHIG INVNSIVSVA TKRWEDSDIF SGKIATARIS YDGSAKILTV 181 VLSYPDGADY ILSHSVDLSK NLPNPIRVGI SASTGANQFL TVYVLSWRFS SALQSTSVNA 241 AMGPEIIRTV V or variants, e.g., conserved variants, or portions thereof that bind alpha GalNAc.

Contemplated saccharide binding proteins include Griffonia simplicifolia lectin isoform II (SEQ ID NO:6) MAKSTAKPNF SLLLPILISL FLFQLNRVKS ADTVCFTSTS FGKDVSDLTLQ or variants, e.g., conserved variants, or portions thereof that bind N-acetylglucosamine.

Contemplated saccharide binding proteins include Erythrina cristagalli lectin (SEQ ID NO: 7) VETISFSFSE FEPGNNDLTL QGAAIITQSG VLQLTKINQN GMPAWDSTGR TLYTKPVHIW 61 DMTTGTVASF ETRFSFSIEQ PYTRPLPADG LVFFMGPTKS KPAQGYGYLG VFNNSKQDNS 121 YQTLAVEFDT FSNPWDPPQV PHIGIDVNSI RSIKTQPFQL DNGQVANVVI KYDASSKILL 181 AVLVYPSSGA IYTIAEIVDV KQVLPEWVDV GLSGATGAQR DAAETHDVYS WSFHASLPET 241 ND or variants, e.g., conserved variants, or fragments that bind galactosyl (3-1,4) N-acetylglucosamine

The polymer may also be covalently attached to polypeptides that provide access to crossing the blood brain barrier. For example, Xin et al., Biomaterials, 2011, 1-13, hereby incorporated by reference, have also disclosed peptide-conjugated poly(ethylene glycol)-co-poly(e-caprolactone) nanoparticles act as a targeting drug delivery system for brain glioma. It is disclosed that angiopep protein (SEQ ID NO:1)TFFYGGSRGKRNNFKTEEY, improves the ability for the polymers to cross the blood-brain barrier.

### EXPERIMENTAL

### EXAMPLE 1

### Cell Sorting of CD133⁺ GBM-Derived CSCs Using Magnetic Bead Separation

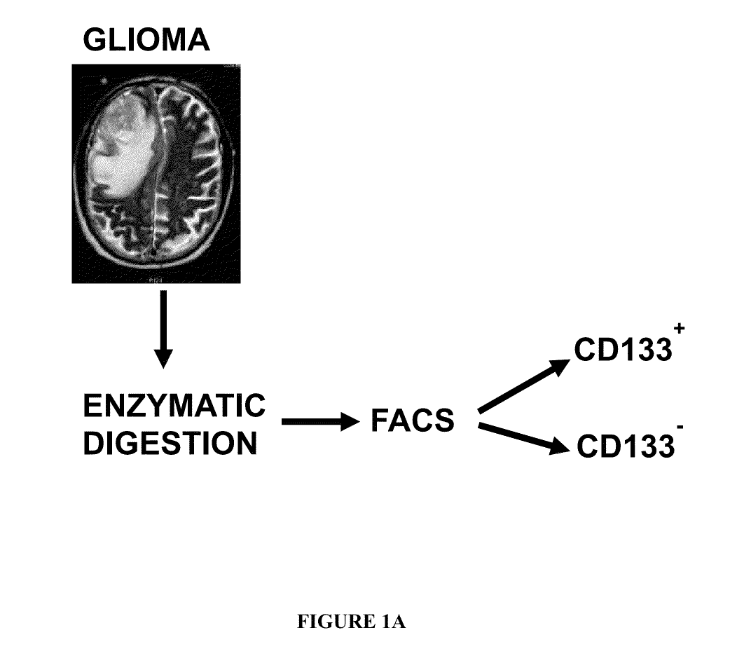

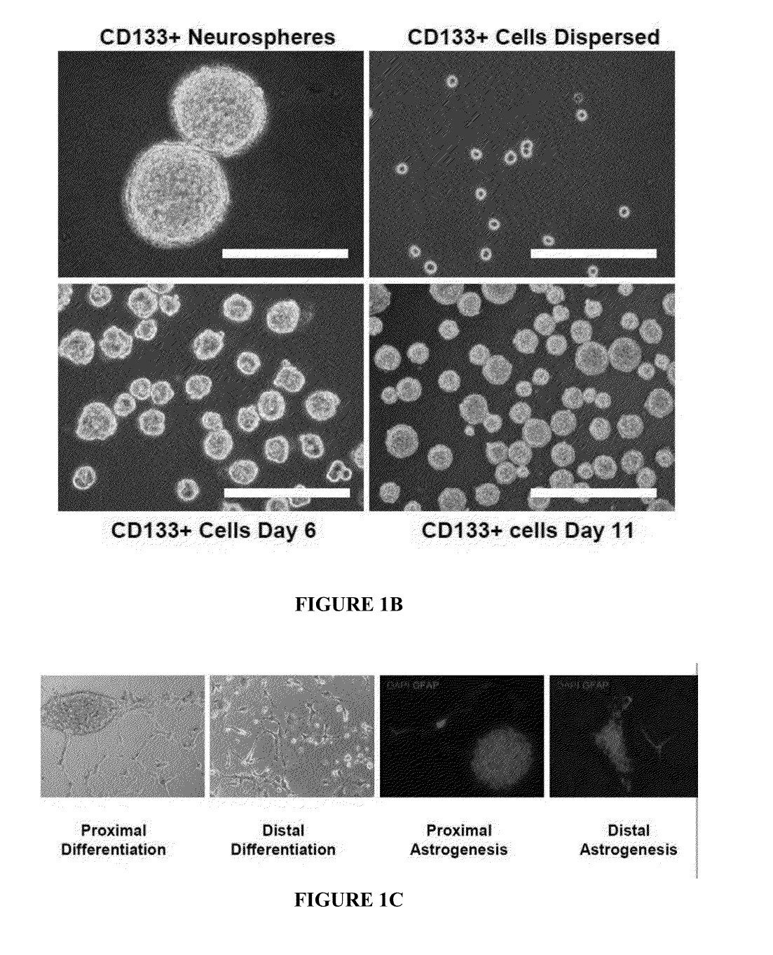

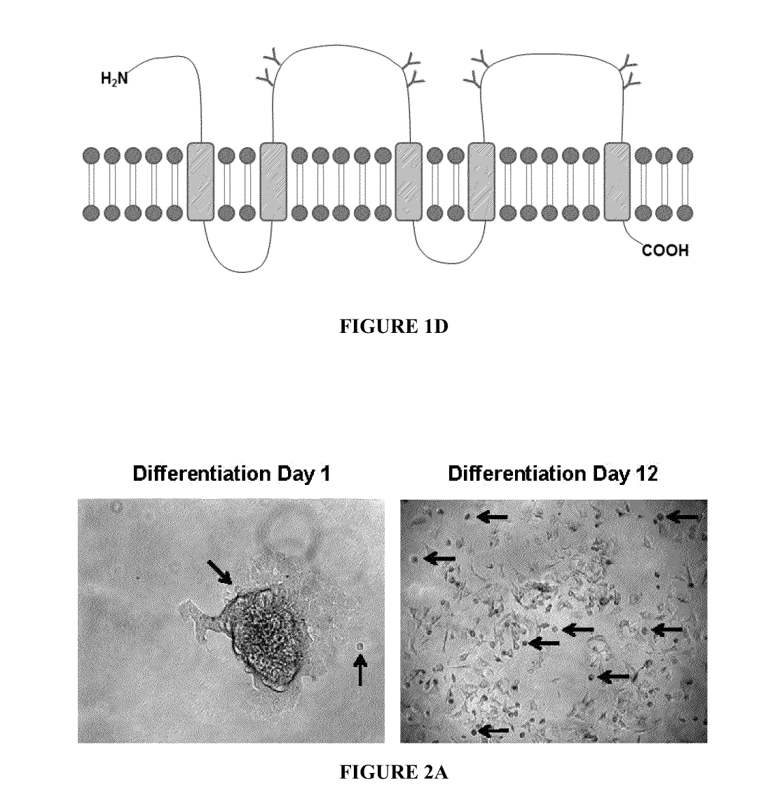

Enzymatically digested tumor, while in single cell suspension was labeled with biotinylated CD133 antibody. Metallic beads coupled with strepavidin were added to the mixture and cell which were positive for CD133 were successfully separated from the total population. It has been reported that CD133⁺ GBM-derived CSCs make up approximately 0.1%-10% of the tumor, however the average is <1%. After multiple sorts, our CD133⁺ positive population averaged 40-50% of the total population collected, which was less than 1% of the total tumor. The CD133⁺ cells formed neurospheres and were grown until they reached 150 μM in diameter, at which they can be passaged or frozen and stored. Afterwards, the neurospheres were plated onto chamber slides where they successfully differentiated into TUJ-1 positive neuronal lineages as well as glial cells (Astrocytes) (FIG. 1). This result shows that we are able to isolate CD133⁺ GBM-derived CSCs from our tumor and these isolated cells are capable of differentiating in neuronal and glial cell lineages.

### EXAMPLE 2

### Lectins Identify GalNAc and GlcNAc on the Surface of CD133⁺ GBM-CSCs

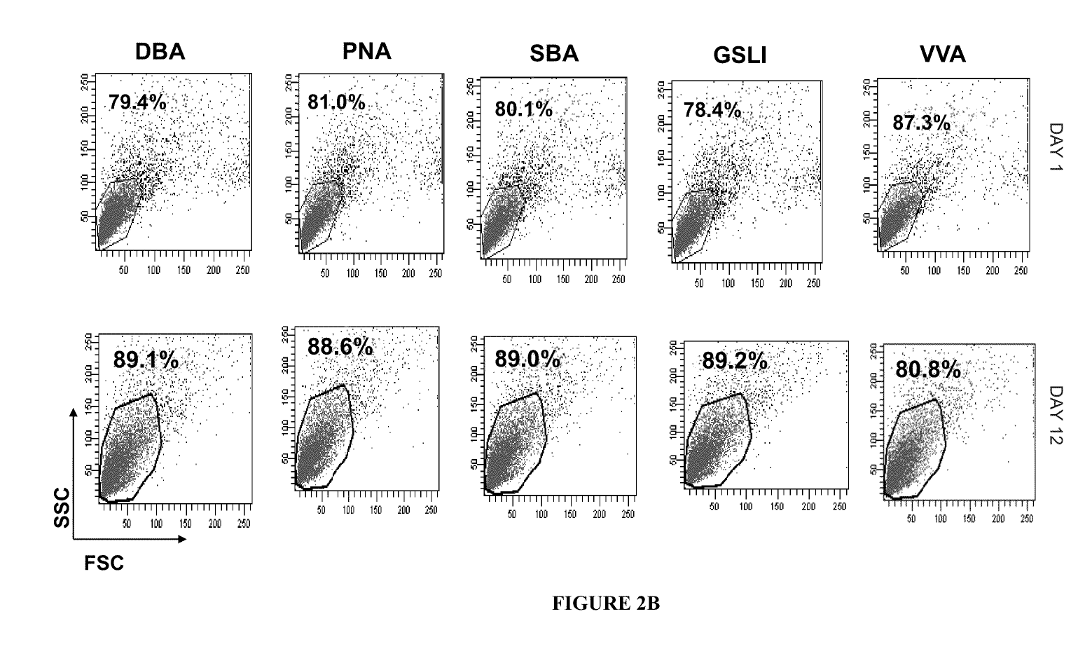

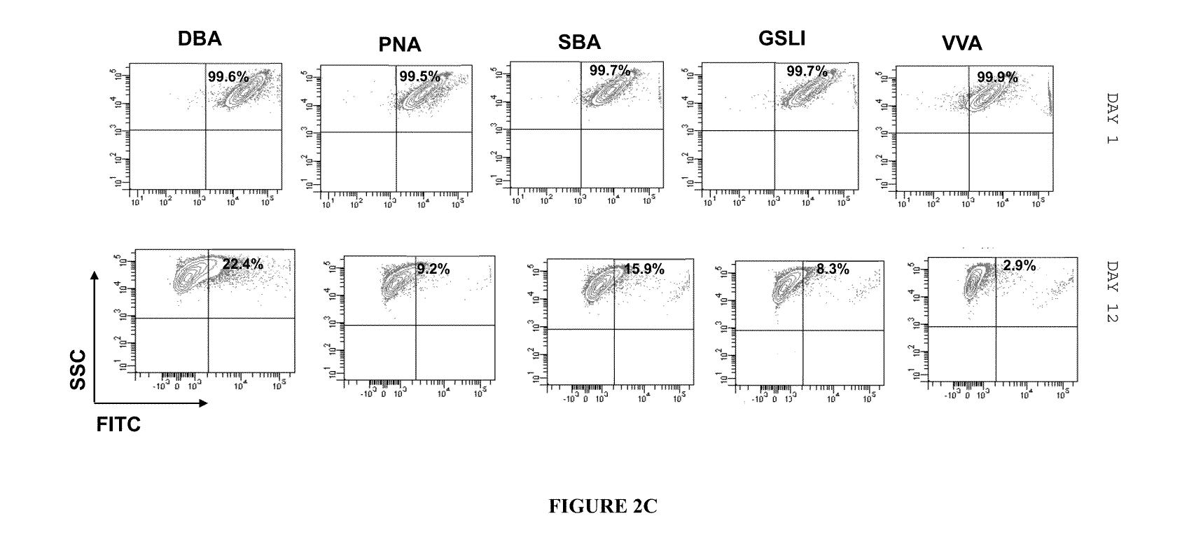

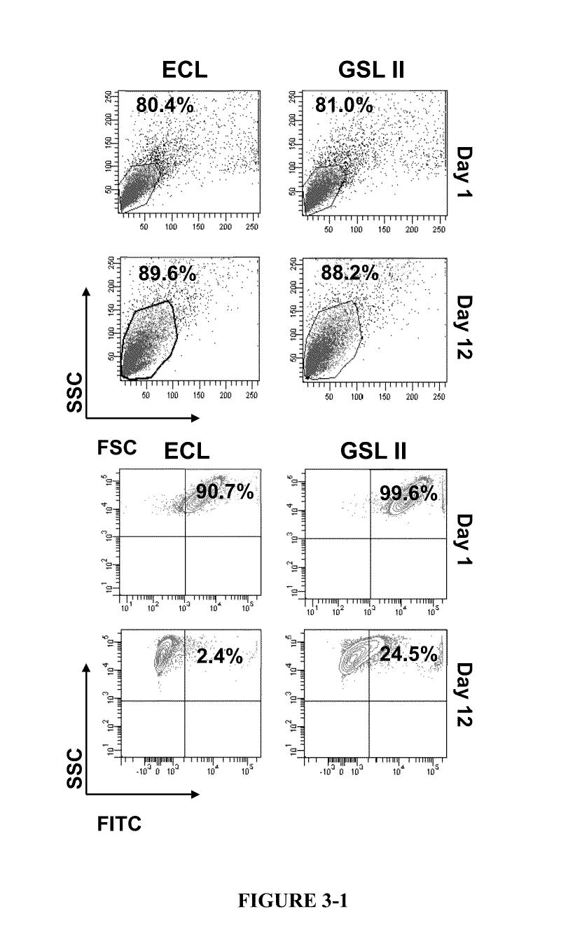

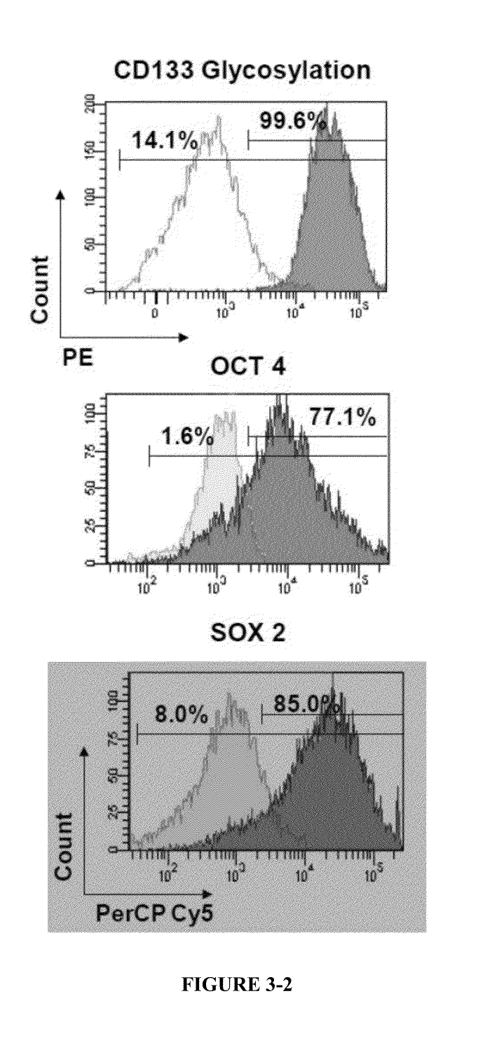

The fact that lectin moieties recognize carbohydrates located on the surface of cells makes them a great tool to identify changes in glycosylation patterning during differentiation. We used a panel of 20 lectins to study the patterning of CD133⁺ GBM-CSCs surface glycosylation during differentiation (Table 1). Neurospheres derived from CD133⁺ GBM-CSCs were grown as mentioned above and were plated onto polyornithin and laminin coated tissue culture grade plastics. Within 10 hours the neuroshperes adhered and neurite formations were observed. Differentiation continued for 12 days, and harvested cells (day 1 and day 12) were labeled with our panel of lectins (FIG. 2A). A quantitative evaluation of the cell populations show lectins DBA, PNA, SBA, GSL I, and VVA, all of which recognize the glycan moiety GalNAc, highly recognized undifferentiated GBM-CSCs. However, this recognition markedly decreased after 12 days of differentiation (FIGS. 2A and 2B). Similarly lectins ECL and GSL II, which recognize the glycan moiety GlcNAc, highly recognized undifferentiated GBM-CSCs. This recognition noticeably decreased after 12 days of differentiation (FIGS. 3A and 3B). Furthermore, the recognition and decrease in recognition of GalNAc and GlcNAc coincide with the expression of GBM-CSC marker CD133-glycosylation (CD133-G) and general stem cell markers OCT 4 and SOX2 (FIG. 3C). OCT 4 and SOX2 have been well studied in GBMs and are accepted indicators of sternness in GBM cell lines. This result shows that specific lectins can identify glycans GalNAc and GlcNAc which are uniquely expressed on the surface of GBM-CSCs. This result is derived through non-destructive techniques and correlate to the invasive (destructive) analysis of general stem cell markers OCT 4 and SOX 2.

### EXAMPLE 3

### Fluorescently Labeled DBA and Cell Surface Glycan GalNAc can be Used to Sort GBM-CSCs to Establish New Cell Lines and have Less Proliferation but Greater Apoptosis

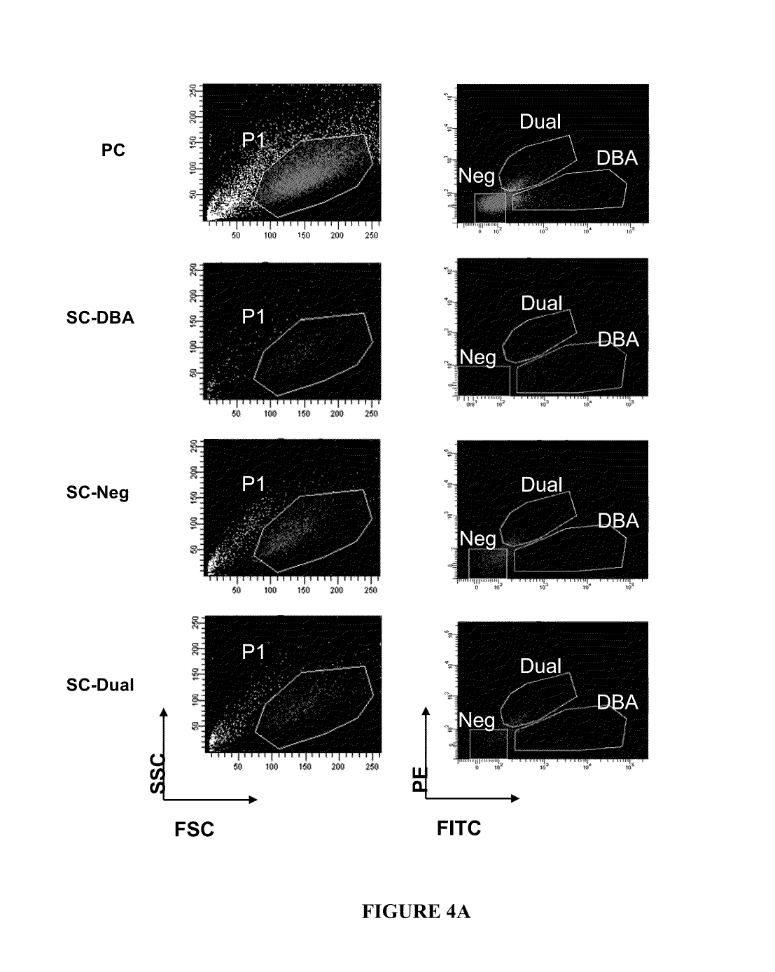

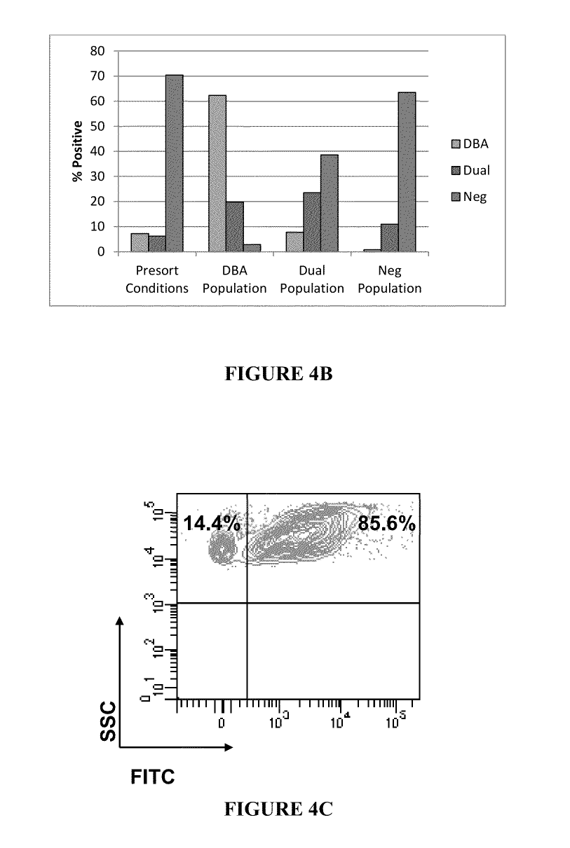

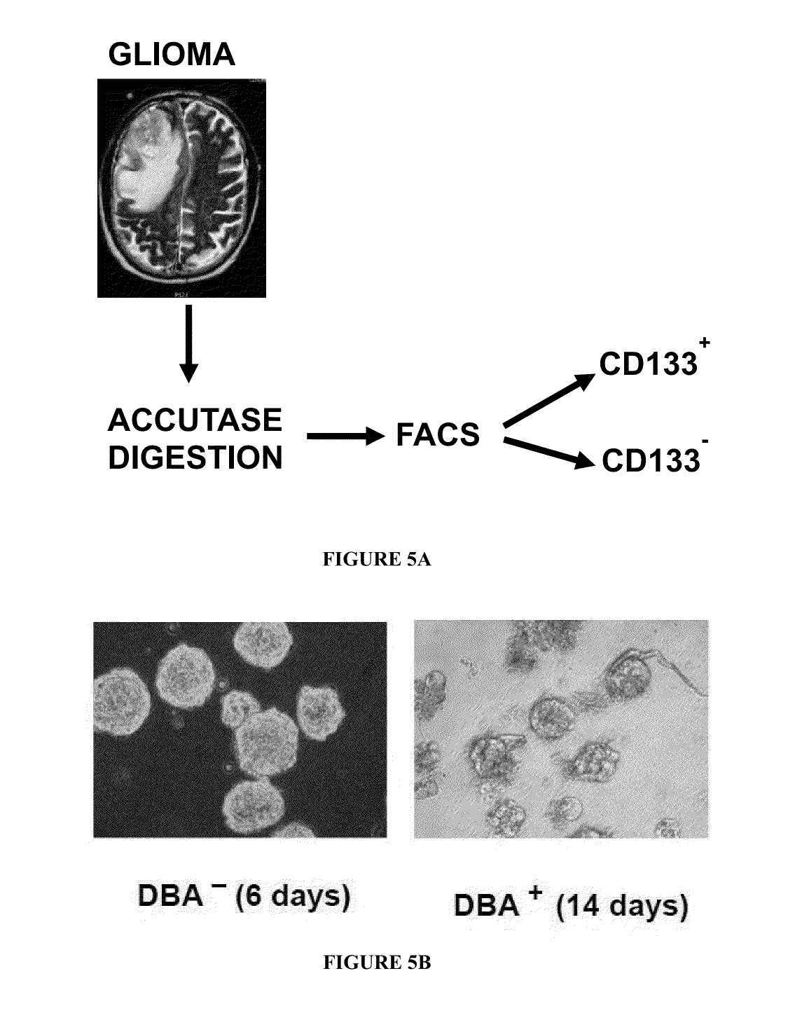

GBM cells, in single cell suspension, were treated with fluorescently labeled DBA lectin and two individual technologies were employed to separate positively labeled cells from the parent population. BD FACS Aria cell sorter successfully separated and quantitatively analyzed positively labeled cells for DBA from non-labeled (Neg. Population) and Dual labeled cells (Fluorescently labeled DBA and fluorescently labeled actin antibody) (FIGS. 4A and 4B). DBA has specificity for GalNAc and can be competed off of cells using 200 mM free (unbound) GalNAc (FIG. 4C), suggesting that non-specific interaction between DBA lectin and proteins or random glycans is very unlikely. Contamination (bacterial) being of great concern, a second, more sterile, method was developed using the autoMACS Pro Separator (Miltenyi Biotec). This benchtop automated magnetic cell sorter was used in a sterile tissue culture cabinet and has a proven record for the isolation of virtually any cell type from any species. Using auto MACS magnetic beads which recognized fluorescent labeling one is able to separate DBA positive labeled cells from non-labeled. See FIG. 5A.

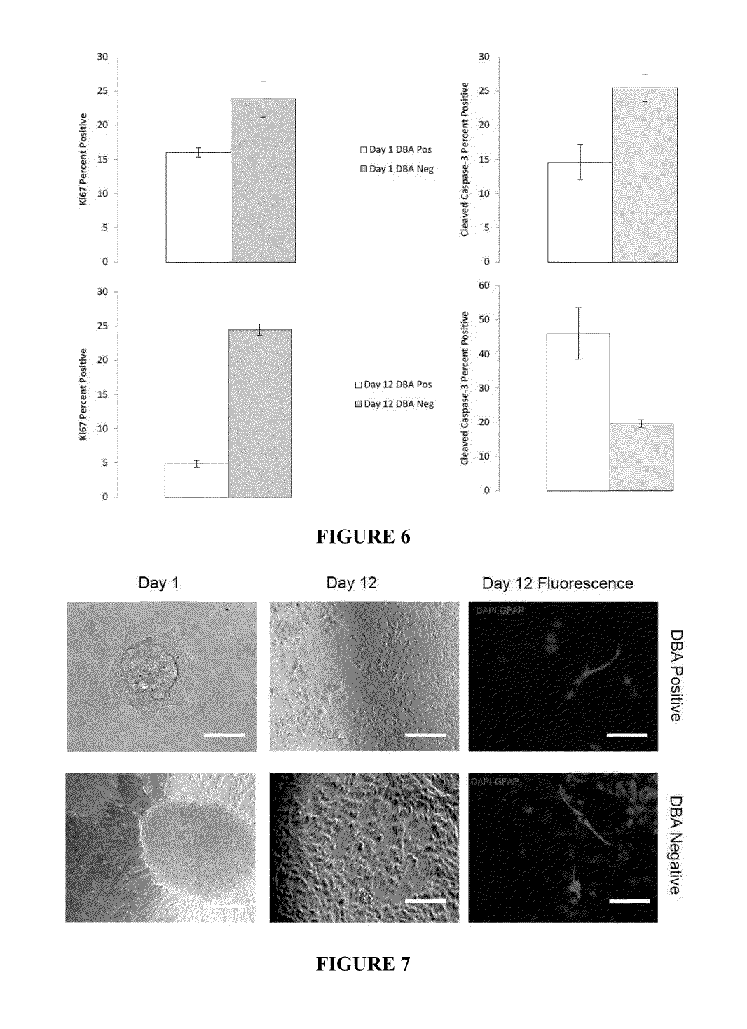

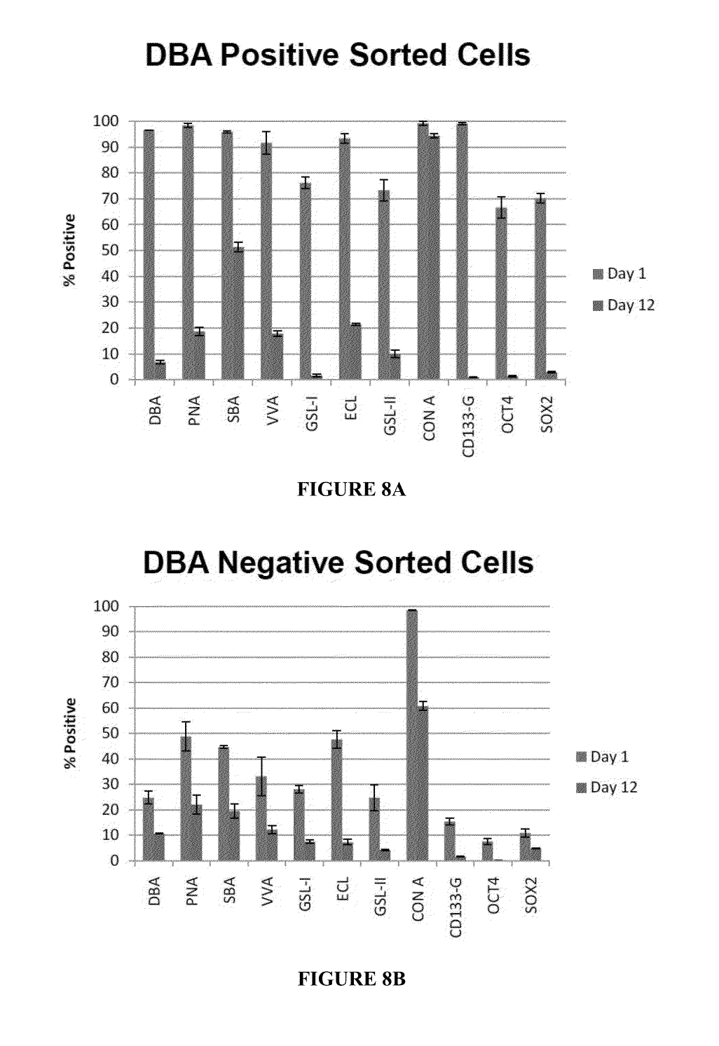

DBA lectin positive cells made up less than 1% of the total population and phenotypically appeared to proliferate at a much slower rate than the DBA negative population. See FIG. 5B. Measuring the diameter of the neurospheres confirmed that at greater than twice the time course, cells positively sorted for DBA were smaller suggesting slower proliferation or increased cell death (FIG. 5C). Ki67 positive staining is a hallmark of proliferation and day 1 DBA negative populations showed higher Ki67 antibody reactivity (23.8%) versus the DBA positive population (16%) (FIG. 6). This trend continued throughout differentiation and by day 12, 24.5% of DBA negative sorted cells were Ki67 positive, meanwhile DBA positive sorted cells showed a reduction in reactivity to KI67 (4.9%). Cleaved caspase 3, an indicator of apoptosis, is elevated in the DBA negative population at day 1 (25.5) versus the DBA positive population (14.6), however, by day 12 apotosis appears to increase in the DBA positive population to almost half (46%) while the DBA negative population shows a decrease in cell death (19.6%) (FIG. 6). Increased proliferation in DBA negative populations can be visualized via microscopy at day 1 and day 12 (FIG. 7). At day 12 the DBA negative population appears to be denser than the DBA positive population (FIG. 7), however, both populations are able to produce GFAP positive cells (Astrocytes) identical to the CD133 positive sorted cells (FIG. 7). Positively sorted DBA cells show high reactivity for lectins DBA, PNA, SBA, VVA, GSL-I, which recognize GalNAc, and decrease by day 12 (FIG. 8A). GSL-II and ECL, which recognize GlcNAc, are highly reactive as well in the DBA positive sorted cells and also decreased by day 12 (FIG. 8A). DBA negative sorted cells contradict the aforementioned, and show low reaction towards GalNAc and GlcNAc recognizing lectins and this continues to decrease by day 12 (FIG. 8B). Con A remains high throughout in both populations and serves as a positive control (FIGS. 8A and 8B). However, GBM-CSC marker CD133-G and general stem cell markers OCT 4 and SOX 2 are high in the DBA positive sorted cells, and similar to DBA reactivity, decreases substantially by day 12 of differentiation (FIG. 8A). However, in the DBA negative population these stem cell markers uniformly are low at day one and are almost non-existent by day 12 (FIG. 8A). Until now CD133 has been considered the only marker for CSCs derived from GBM brain tumors. However, using cell sorting technology, DBA lectin, which recognizes GalNAc, we were able to separate CD133⁺ cells from CD133− cells. Also, this data shows proliferation is higher in GBM cells that are more differentiated, however during differentiation GBM-CSC become more apoptotic. This data shows that lectins that recognize GalNAc can be used to identify and sort GBM-CSCs.

### EXAMPLE 4

### DBA Lectin Sorted GBM-CSCs Produce High Grade Gliomas in NOD/SCID Mice

CD133+ (CTB/CD133+) sorted GBM-CSCs were intracranially implanted (1×10⁵) in NOD/SCID mice to demonstrate tumorigenicity. At six months, an infiltrative high grade glioma was noted confirming the orthotopic tumorigenicity of these cells. To determine the tumorigenicity of DBA+ sorted cells (CTB-1/DBA+), 1×10⁴ cells were implanted into the flank of NOD/SCID mice.

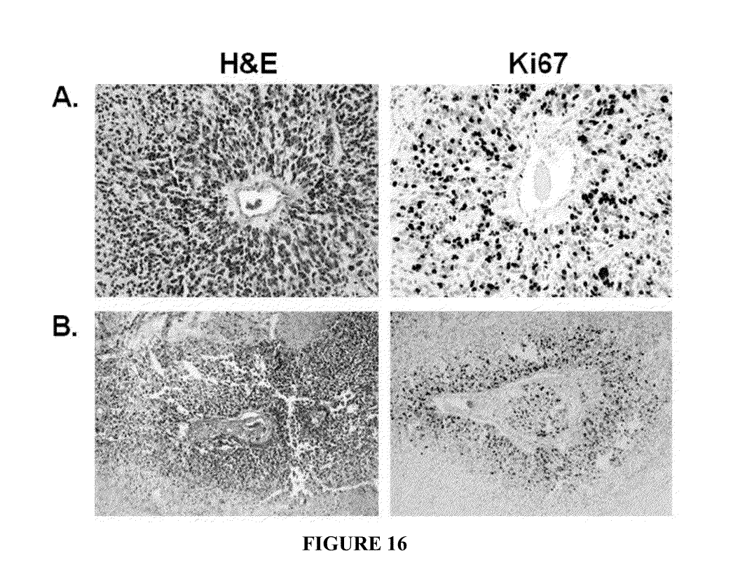

These CTB-1/DBA+ cells produced a high-grade neoplasm with features of a glioblastoma, including high cell density, an infiltrative pattern and angiogenesis (FIGS. 16A and B (H+E stain)). DBA+ neurosphere derived tumors also display high immune-reactivity with the Ki67 antibody confirming their high proliferation rate. See FIG. 16.

### EXAMPLE 5

### GalNAc is a Unique Structure and is Not Part of the CD133

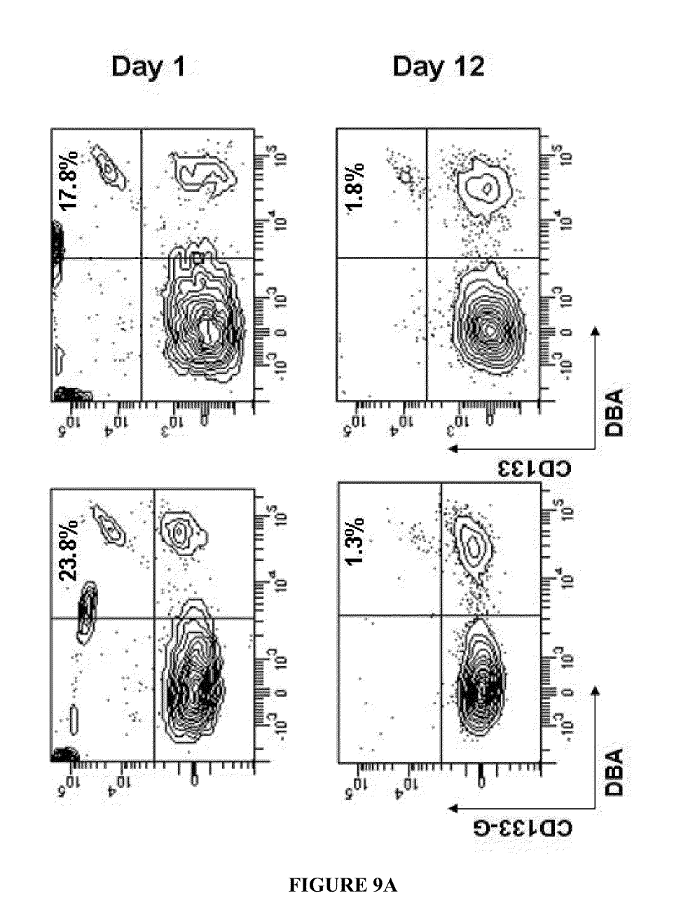

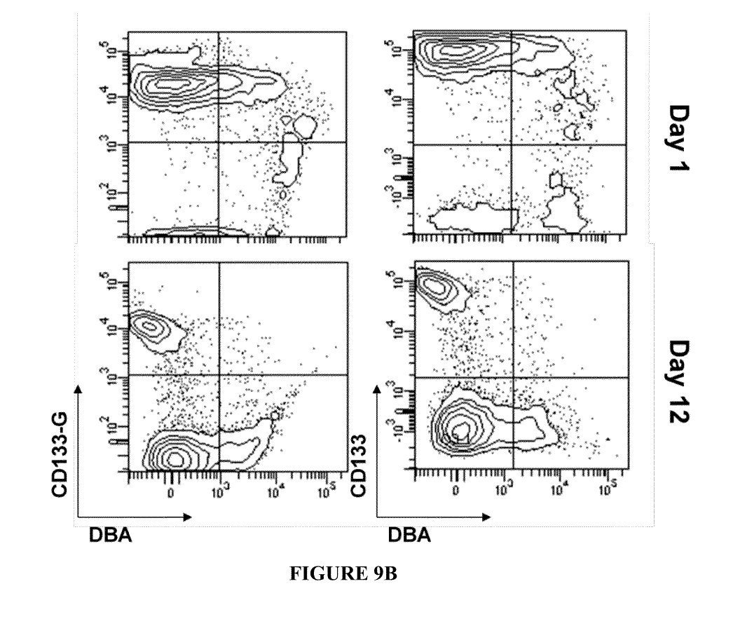

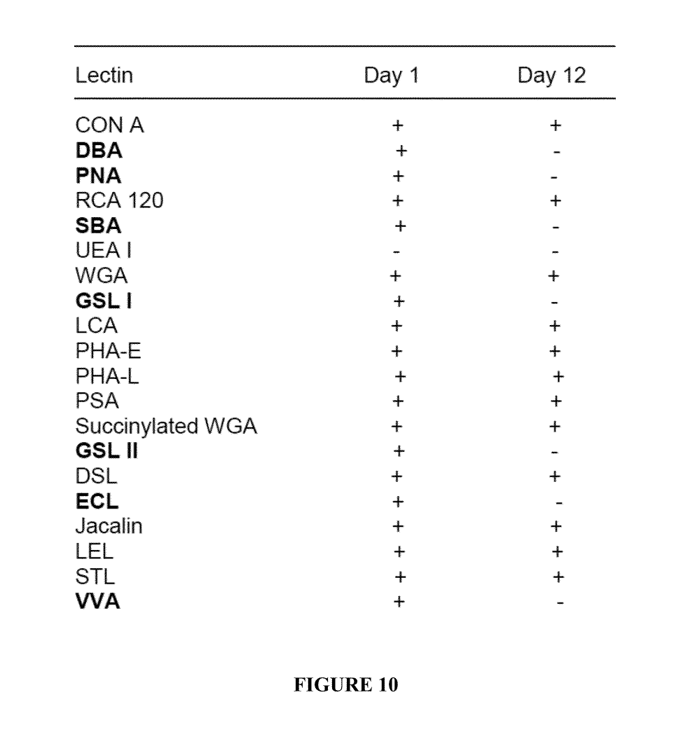

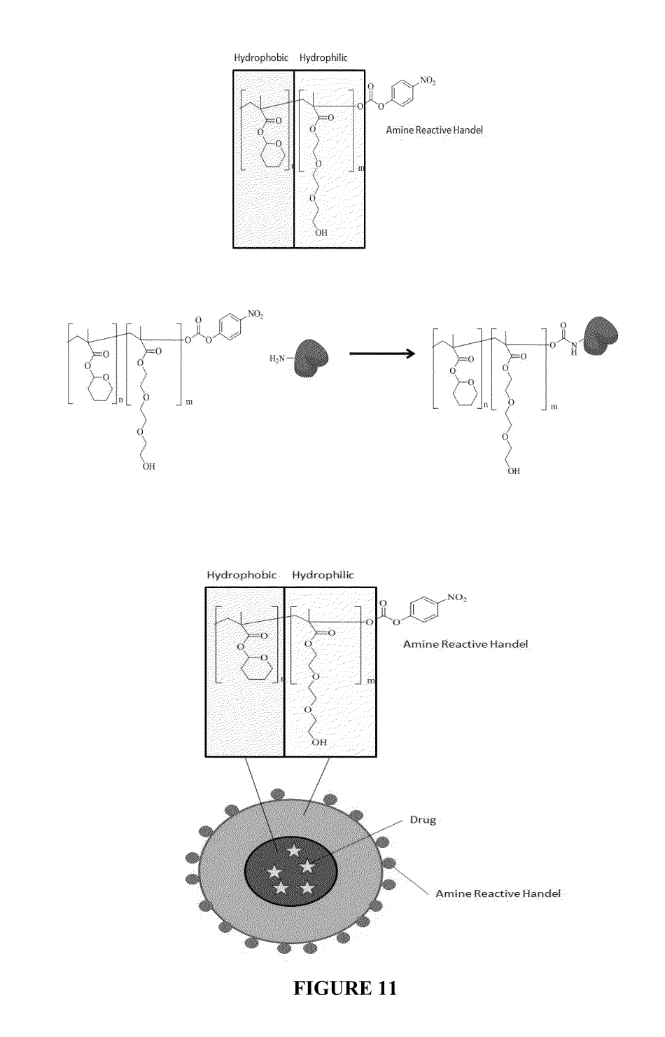

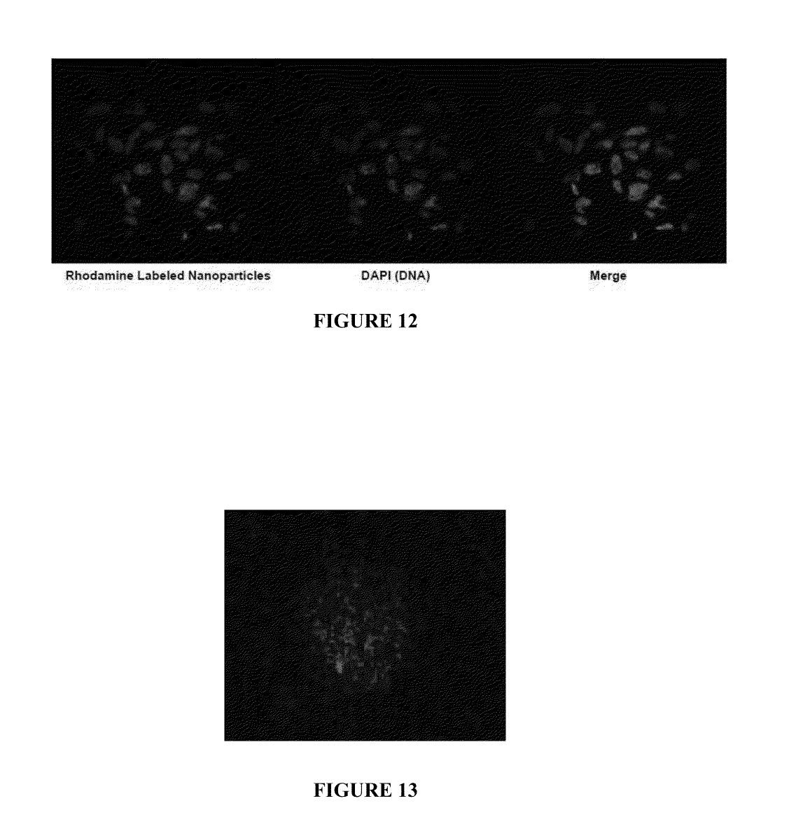

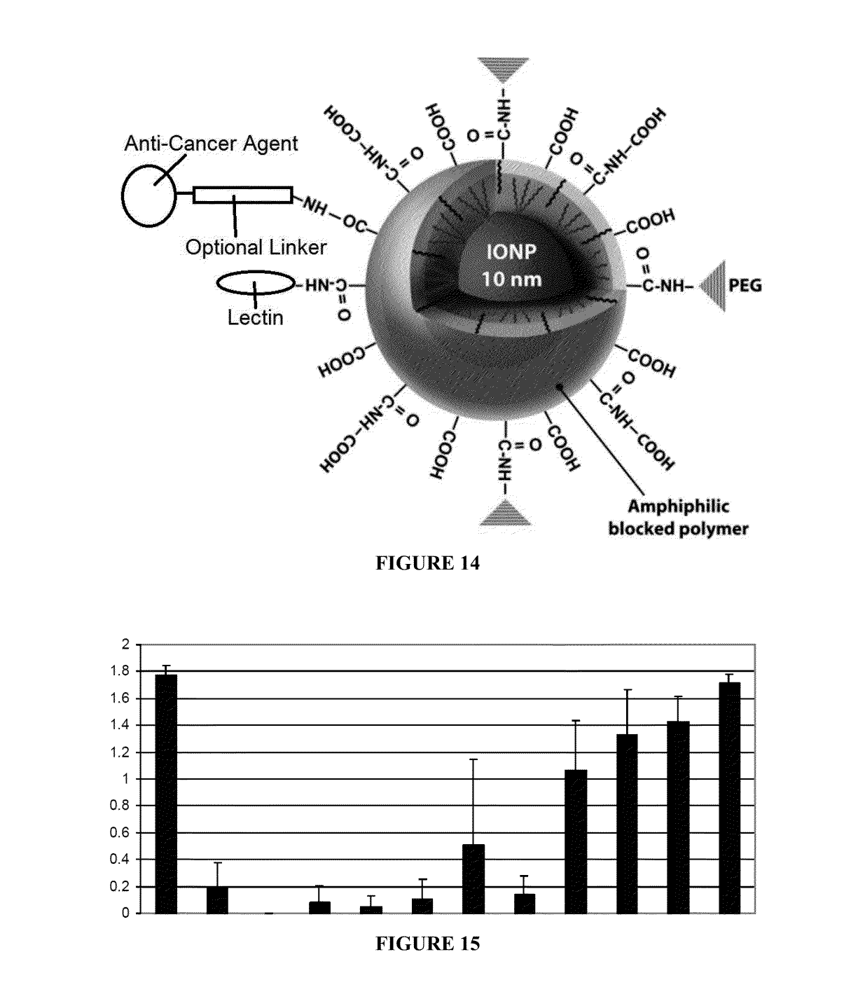

FIG. 9 shows DBA sorted negative (A) and positive (B) cells and the individual populations of DBA (GalNAc) positive, CD133-G and CD133 populations. Clearly, the GalNAC positive cells are a distinct population and this marker can be used to isolate and sort stem cells.

### EXAMPLE 6

### Nanoparticle Preparation

Nanoparticles with sizes of approximately 200 nm are formulated by first, suspending the solid block copolymer in tetrahydrofuran (THF) at 10 mg per ml of solvent. This organic phase is then added drop-wise to a vigorously stirred 0.01 M pH 6.5 PBS solution. The resulting organic in water emulsion is then allowed to stiff for 30 min to allow the particles to set-up. The emulsion is then stirred under reduced pressure via a rotary evaporator to remover remaining THF. The aqueous solution containing the now hardened particles is then added to two equivalents of a stirred 0.1 M pH 7.4 PBS solution. Lectins or BSA are then added to this solution and react to the amine reactive groups on the particles. The reaction is allowed to continue for 2 hours. Protein conjugated particles are washed of any free protein via centrifugation. The particles are then washed 2× via resuspention in PBS and subsequent centrifugation. Finally, the particles are lyophilized for long term storage.

### EXAMPLE 7

### Lectins Conjugated in Combination with Cetuximab to Iron Oxide Nanoparticles Kill U87wtEGFR Glioblastoma Cells

Polymer coated iron oxide nanoparticles with terminal carboxylic acid groups may be obtained from Ocean Nanotech. Lectins DOLICHOS BIFLORUS AGGLUTININ (DBA) or GRIFFONIA SIMLICIFOLIA II (GSLII) in combination with Cetuximab are conjugated to iron oxide nanoparticles through a polymer coat that covers an iron oxide nanoparticle (IONP). Any polypeptide containing a primary amine group may be conjugated to the IONPs using following coupling procedure or as appropriately modified. A solution of Lectin/Cetuximab is mixed with an IONP comprising carboxy terminated polymer that has been activated with an EDAC/NHS mixture.

The reduction of tetrazolium salts is a reliable way to examine cell proliferation. The yellow tetrazolium MTT (3-(4,5-dimethylthiazolyl-2)-2,5-diphenyltetrazolium bromide) is reduced by metabolically active cells to generate reducing equivalents such as NADH and NADPH. The resulting intracellular purple formazan is quantified by spectrophotometric means. The MTT Cell Proliferation Assay measures the cell proliferation rate and conversely, when metabolic events lead to apoptosis or necrosis, the reduction in cell viability. FIG. 15 shows data on lectins conjugated in combination with cetuximab, a EGFR antibody, to the polymer coat surrounding an iron oxide nanoparticle. The results suggest IONPs conjugated with cetuximab in combination with DBA are preferred.

1. A conjugate comprising:
a) a saccharide binding moiety;
b) a polymer; and
c) a therapeutic agent;
wherein the saccharide binding moiety is covalently attached to the polymer.

2. The conjugate of claim 1, wherein the saccharide binding moiety is a lectin selected from the group consisting of concanavalin A (CON A), dolichos biflorus agglutinin (DBA), peanut agglutinin (PNA), ricinus communis agglutinin I (RCA 120), soybean agglutinin (SBA), ulex europaeus agglutinin I (UEA I), wheat germ agglutinin (WGA), griffonia simplicifolia lectin I (GSL I), lens culinaris agglutinin (LCA), phaseolus vulgaris erythroagglutinin (PHA-E), phaseolus vulgaris leucoagglutinin (PHA-L), pisum sativum agglutinin (PSA), griffonia simplicifolia lectin II (GSL II), datura stramonium lectin (DSL), erythrina cristagalli lectin (ECL), Jacalin, lycopersicon esculentum lectin (LEL), solanum tuberosum lectin (STL), and vicia villosa lectin (VVA).

3. The conjugate of claim 1, wherein the saccharide binding moiety binds a saccharide or polysaccharide selected from the group consisting of α- or β-linked N-acetylgalactosamine, branched and terminal α-linked mannose, α-linked N-acetylgalactosamine, galactosyl (β-1,3)N-acetylgalactosamine, oligosaccharides ending in galactose, terminal α- or β-linked N-acetylgalactosamine, α-linked fucose residues, terminal N-acetylglucosamine or chitobiose, α-N-acetylgalactosamine and α-galactose residues, α-linked mannose residues, galactose, α-linked mannose residues, α- or β-linked N-acetylglucosamine, N-acetyllactosamine, galactosyl (β-1,4)N-acetylglucosamine, galactosyl(β-1,3)N-acetylgalactosamine, N-acetylglucosamine, N-acetylglucosamine and N-acetylmuramic acid.

4. The conjugate of claim 1, wherein the polymer forms a particle comprising an outer hydrophilic coat.

5. The conjugate of claim 1, wherein the polymer comprises lactone containing monomers and ethylene glycol containing monomers.

6. The conjugate of claim 1, wherein the therapeutic agent is an anticancer agent.

7. The conjugate of claim 1, wherein polymer surrounds the therapeutic agent.

8. The conjugate of claim 1, wherein the therapeutic agent is conjugated to the polymer through a biodegradable bond.

9. The conjugate of claim 1, wherein the therapeutic agent is temozolomide, bevacizumab, doxorubicin, hydroxydaunorubicin, bleomycin, dactinomycin, vinblastine, dacarbazine, mechlorethamine, cyclophosphamide, etoposide, teniposide, vincristine, prednisone, platinum agent (cisplatin, carboplatin, oxaliplatin), fluorouracil, folinic acid, carmustine, rituximab, methotrexate, procarbazine, epirubicin, irinotecan, ifosfamide, chlorambucil, lomustine, leucovorin, fludarabine, thalidomide, dexamethasone, docetaxel, anastrozole, topotecan, combretastatin, or combretastatin A-4 phosphate.

10. A composition comprising a conjugate of claim 1, wherein the polymer surrounds a particle.

11. The composition of claim 10, wherein the particle has a diameter of between about 200 and 5 nm.

12. The composition of claim 10, wherein the particle is metal particle comprising an iron oxide particle, elemental iron coated with iron oxide, gold, silver, a quantum dot, or bismuth encapsulated in a phospholipid core.

13. The composition of claim 10, wherein the therapeutic agent is an EGFR antibody.

14. The composition of claim 13, wherein the therapeutic agent is cetuximab.

15. The composition of claim 14, wherein the saccharide binding moiety is DBA.

16. A method of treating cancer comprising administering a composition comprising conjugate of claim 1 to a subject diagnosed with cancer.

17. The method of claim 16, wherein the subject is diagnosed with a brain tumor.

18. The method of claim 16, wherein the subject has previously undergone surgical removal of a tumor or radiation therapy.

19. The method of claim 16, wherein the subject is administered the composition in combination with radiation therapy.

20. The method of claim 16, wherein the composition is administered orally, by injection, convection enhance delivery, or intracerebrally
